(19) United States
(12) Patent Application Publication
(10) Pub. No.: US 2023/0063977 A1
(43) Pub. Date: Mar. 2, 2023

(54) TRANSCATHETER SOFT ROBOT

(71) Applicant: Kennesaw State University Research And Service Foundation, Inc., Kennesaw, GA (US)

(72) Inventors: Amir Ali Amiri Moghadam, Marietta, GA (US); Ayse Tekes, Marietta, GA (US)

(21) Appl. No.: 17/900,175

(22) Filed: Aug. 31, 2022

Related U.S. Application Data
(60) Provisional application No. 63/239,198, filed on Aug. 31, 2021.

(51) Int. Cl. A61B 34/30; A61F 2/24

(52) U.S. Cl. CPC A61B 34/30; A61F 2/243; A61F 2/2436; A61B 2034/304; A61B 2034/303

(57) ABSTRACT

A device comprising a first stent, a plurality of flexible links coupled to the first stent, a plurality of compliant links, each compliant link coupled to at least one of the flexible links, a second stent coupled to the plurality of compliant links, wherein the plurality of flexible links and the plurality of compliant links are configured to steer one or more of the first stent and the second stent in a plurality of degrees of freedom.

### CROSS-REFERENCE TO RELATED APPLICATIONS

This application claims priority to U.S. Provisional Patent Application No. 63/239,198, filed Aug. 31, 2021, the disclosure of which is hereby incorporated by reference in its entirety.

### BACKGROUND

Cardiac diseases are the major cause of death in the United States. Each year about 655,000 Americans die because of cardiac disease which is one of every four reported deaths. There are shortcomings of current catheter technology for treatment and diagnosis of Congenital Heart Disease, and Women's Heart Disease such as intracardiac echocardiography (ICE), atrial fibrillation ablation, repair of mitral valve prolapses, and closure of the ventricular septal defect. The existing heart interventions include open heart surgery, where the surgeon cuts the chest and heart open to access the area of interest, robotic assisted heart surgery, where the surgeon accesses the heart though a small incision in the rib cage but then cuts the heart open to access the area of interest, and transcatheter interventions, where the surgeon inserts a catheter into the heart through a vein or an artery in the leg or arm without cutting the heart open. Thus, transcatheter interventions can revolutionize the treatment of cardiac disease by causing significantly less trauma to the patients.

Compared to the open-heart and robotic assisted heart surgery, transcatheter interventions dramatically minimize the patient recovery time and also reduce the risk of infection, anesthesia and average length of stay in the hospital. Additionally, open-heart and robotic assisted surgery may be high-risk options for many elderly patients and people with diabetes, and in general they are more expensive. Furthermore, for patients at intermediate surgical risk, the overall long-term cost of transcatheter aortic valve replacement (TAVR) is projected to be $8,000-$10,000 less than the surgical aortic valve replacement (SAVR). Therefore, transcatheter intervention is considered the preferred method from both the clinical and the economical perspective.

As noted above, transcatheter interventions are the treatment of choice because they cause the least amount of trauma to patients. However, transcatheter interventions face certain challenges. For instance, the shape and size of transcatheter devices are limited to the geometry of a vein or an artery (e.g., a small tube). This constraint limits the geometry and structure of transcatheter devices. While steerable transcatheter devices have been developed, these devices generally have the shape of a single steerable tube, and the major difference among them may be the actuation mechanism (e.g., pneumatic, tendon driven, electroactive polymer, and thermal actuator). A single tube catheter design suffers from several shortcomings including limited maneuverability, limited degrees of freedom, positioning accuracy, limited blocking force, torsional wind-up for rolling motion, and buckling for translational motion. For example, there may be no a direct relationship between the motion of the catheter handle and its tip. For instance, if the surgeon rotates the chatter handle by 30° the catheter tip will rotate less than 30° .

The precise steering of the catheter tip inside the heart still remains a challenging task for cardiologists. This may be due to the limitation of the existing devices, the dynamic environment of the heart, and its complex 3D shape. Mal-positioning of transcatheter devices has been reported as a frequent issue in several cardiac interventions such as transcatheter heart valve implantation. Steerable catheters can be divided into two categories based on the generation of force: at the tip or transmitted to the tip. Examples of force generation at the tip include force-generating mechanisms as a result of electrical, thermal, or magnetic actuation. Examples of force transmission to the tip include hydraulic or mechanical cable actuation. Among these technologies, magnetic and mechanical catheters have been used in cardiac interventions. However, magnetic systems are relatively expensive and require major investments to modify the operation rooms. Mechanical systems have limited steering potential. For instance, it may be nearly impossible to track circular curves during a pulmonary vein (PV) isolation procedure. Most of the existing mechanically steerable catheters are comprised of a thin flexible tube that has a single segment tip. These systems do not allow motion in more than two planes (two degrees of freedom (DOF)).

Cardiac intervention in the complex anatomy of the heart requires a high level of dexterity. To solve this issue, more segments can be added to the catheter tip to increase its dexterity or manueverability. However, a catheter with several segments will be less stable and will have a limited blocking force. Moreover, in the existing design of catheters the roll and translational motions may be solely due to the rotation and translation of the catheter handle. Considering the structure of the catheter as a long thin flexible tube and the existence of friction, torsional windup during the roll motion and buckling during the translational motion of the catheter handle may be inevitable. These nonlinear effects result in minimal translation of motion to the distal tip of the catheter which makes effective position control of the catheter difficult. In fact, the effect of the torsional windup is important that clinicians may be using a two-handed technique to roll the catheter handle to increase the efficiency of roll motion and decrease the catheter tip backlash due to the torsional windup. In this technique, a clinician manipulates the handle with one hand and holds the catheter shaft with the other hand to increase the amount of torque which may be exerted to the catheter tip in order to compensate for the effect of torsional wind up.

Various researchers have been working on the development of robotic catheter systems. These developments include an actuation module for position control of a four DOF ultrasound imaging catheter; model-based and model-free control methods for position control of a three DOF steerable catheter; and an automatically controlled three DOF standard cardiac electrophysiology (EP) catheter. However, these developments largely focus on utilizing conventional existing catheter systems which inherently have limited degrees of freedom and suffer from the aforementioned issues of torsional wind-up and buckling for roll and translational motions respectively. Commercially available catheter robots such as Amigo and CorPath utilize standard EP catheters and thus will have limited DOF. Other examples such as the Artisan and EPOCH/V-Drive can only control the position of the catheter but cannot control its orientation.

Thus, there is a need for a steerable catheter with a high level of maneuverability, precise positioning, and sufficient blocking force.

### SUMMARY

A device comprising a first stent, a plurality of flexible links coupled to the first stent, a plurality of compliant links, each compliant link coupled to at least one of the flexible links, a second stent coupled to the plurality of compliant links, wherein the plurality of flexible links and the plurality of compliant links are configured to steer one or more of the first stent and the second stent in a plurality of degrees of freedom.

### BRIEF DESCRIPTION OF THE DRAWINGS

The following drawings show generally, by way of example, but not by way of limitation, various examples discussed in the present disclosure. In the drawings:

FIG. 1 depicts a rigid Delta robot;

FIG. 2 shows a universal joint with 2 degrees of freedom;

FIG. 3 shows a steerable fiber with 2 degrees of freedom;

FIG. 4 illustrates a comparison of a rigid Delta robot and a soft robot;

FIG. 5 illustrates three modes of operation of the soft robot;

FIG. 6 shows details of the soft robot;

FIG. 7 shows 6 steerable fibers inside s delivery sheath;

FIGS. 8A-8B shows a Stewart mechanism;

FIGS. 9A-9B shows details of a converted Stewart mechanism in accordance with the present disclosure;

FIG. 10 shows details of a soft joint;

FIG. 11 shows a soft joint in relaxed state;

FIG. 12 shows a soft joint bent to the left;

FIG. 13 shows a soft joint bent to the right;

FIG. 14 illustrates an example soft joint robot;

FIG. 15 shows a fabricated soft joint robot;

FIG. 16 shows a soft joint robot in operation;

FIG. 17 shows a simulated horizontal trajectory of a soft parallel robot;

FIG. 18 shows a simulated vertical trajectory of a soft parallel robot; and

FIG. 19 shows a simulated circular trajectory of a soft parallel robot.

### DETAILED DESCRIPTION

The present disclosure relates to soft robots designed for use in surgery.

Soft robotics has the potential to revolutionize the interaction of human and robotic systems due to the application of soft and compliant materials in the structure of the robot. As used herein, compliant may mean having a tendency to bend. In a compliant device, the input force, torque, or displacement may be transferred from one point to another point on the device through the deformation of its flexible members rather than the revolute motion between rigid links. Fully compliant mechanisms do not involve any joints and may be designed as a single piece. Semi-compliant mechanisms may comprise both compliant parts and traditional rigid links and joints. Compliant mechanisms can accomplish motions that may be difficult and expensive to achieve by rigid-body devices.

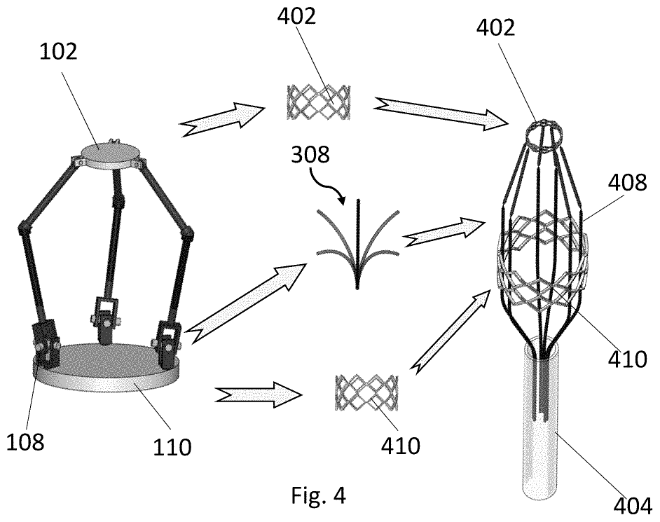

This emerging technology has been utilized in different robotic systems including medical, assistive, and search and rescue robots. There is an increasing demand for collaborative robotic systems that can work in close proximity to humans. While collaborative medical robots and industrial robots can have a positive impact on daily life, safety remains a concern in the application of such robotic systems. The application of external force sensor and monitoring systems can improve the safety of robotic systems. However, these systems cannot guarantee the safety of collaborative robots. Thus, the application of soft, compliant, and lightweight materials in the structure of the robot may be critical to minimize the impact forces due to collision. Compliance in robotic systems can be achieved through active or passive compliance. The active compliance in the rigid robotic systems may be based on the application of sensory force and torque data to control the position of the end-effector. Thus, this method is complex, expensive, and requires a fast real-time response which is hard to achieve. Alternatively, passive compliance can be realized by integrating soft and compliant joints and links in the structure of the robot. Therefore, a passive compliance system can naturally deform in response to external loads without requiring any sensory data or control system. As a result, these systems can be designed properly to safely interact with humans. However, passive compliance systems generally have lower accuracy and complex dynamics. Typically, soft and compliant robots have a serial or hybrid structure. This structure limits the output force and accuracy of the soft robots. Application of parallel structures in the design of soft robots can enhance these characteristics. One way to categorize the existing soft parallel robots may be based on the application of soft links or soft joints in the structure of the robot. The soft robot described herein may be analogous to the rigid Delta robot and may include three soft active links which may be connected to passive links and the robot platforms employing soft joints. FIG. 4 compares the soft parallel robot with an existing rigid robot.

Soft robotic technology may be useful for a novel transcatheter robotic system for intracardiac interventions. Such a robotic catheter system must have six degrees of freedom (DOF) to improve the maneuverability of the catheter and a parallel structure to provide more control on its overall stiffness and blocking force. Moreover, the application of closed-loop kinematic chains in the parallel structure of the robot results in rolling and translational motions that may be independent of the rotation and translation of the catheter handle. Therefore, the effect of torsional windup and buckling in the roll, and translational motions of the catheter may be eliminated. A single tube design enables moving to a novel design of a soft parallel robot. In transcatheter soft robot design, the roll motion can also be generated independently from the rotation of the catheter handle which eliminates the effect of torsional windup and improves the accuracy and efficiency of the rotational motion of the catheter tip.

While soft robotics is an emerging field of research, soft parallel robots are among the latest advancements in this field. Typically, soft robots may be made of serial elastomeric arms. However, the application of a parallel structures in the design of a soft robot may be of great interest since such a structure provides more control on the overall stiffness and the payload of the robot. In this disclosure for the first time, the fabrication of a novel 6 DOF soft parallel robot is described.

It is well known that the minimum required DOF for manipulation in 3D spaces is six, i.e., three translational motions (x, y, and z) and three rotational motions (roll, pitch, yaw). Existing steerable catheters can generate 4 DOF, which is not sufficient for effective manipulation in the complex 3D geometry of the heart. A six DOF reconfigurable soft parallel robot has been fabricated and a design which can fit inside a catheter has been developed. Commercial steerable catheters have limited DOF and cannot control both position and orientation of the catheter's tip. However, the robotic system described herein has six DOF and can control both the position and orientation of the catheter tip, which improves the effective dexterity or maneuverability of the catheter tip. Unlike conventional steerable catheters which solely generate rotational and translational motions based on the rotation and translation of the catheter handle the transcatheter soft robot may generate rotational and translational motions independent of rotation and translation of the catheter handle. This may be of significant importance because it eliminates the adverse effects of torsional wind-up and translational buckling and thus, improves the efficiency and accuracy of the catheter's tip motion.

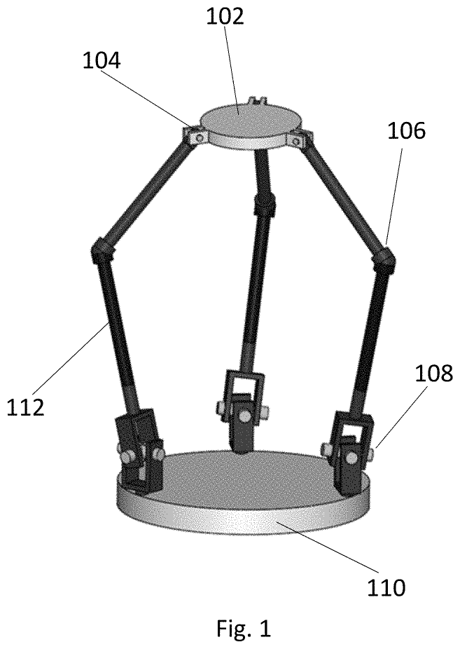

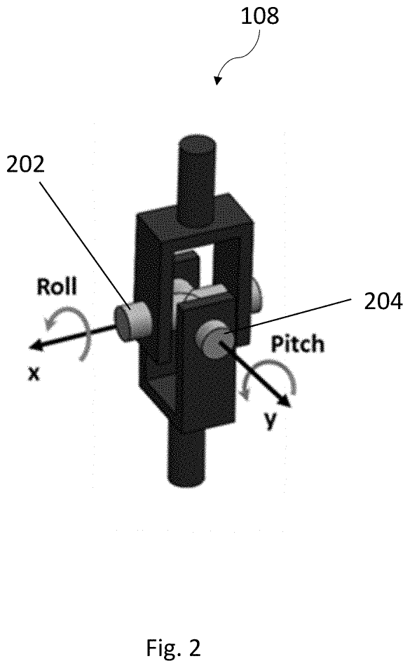

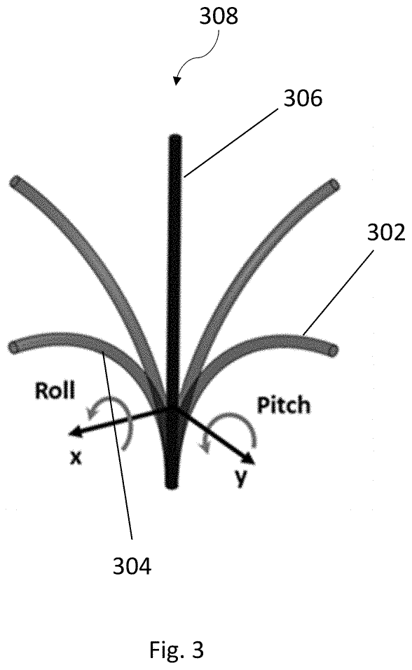

The structure of the soft robot may be similar to that of a rigid three Universal-Spherical-Revolute (3USR) parallel robot, as is shown in FIG. 1. FIG. 1 shows a 3USR robot with three universal joints 108, a rigid bottom platform 110, a rigid top platform 102, legs 112, a top platform joint 104, and a flexure joint 106. FIGS. 2-9 depict a soft robot according to embodiments of the disclosure. The soft transcatheter robot may comprise three soft steerable catheters (where each catheter has 2 DOF) as shown in FIGS. 2-3. Each of the universal joints may move in two angular directions as shown in FIG. 2. FIG. 2 illustrates details of a universal joint 108 with two angular degrees of freedom. One degree of freedom is to rotate about the x-axis 202, called roll. A second degree of freedom is to rotate about the y-axis 204, called pitch. FIG. 3 demonstrates how a controllable or steerable fiber 308 may be employed to achieve similar motions as the universal joint. FIG. 3 shows the fiber in a resting state 306. The fiber bends perpendicular to the y-axis to achieve roll 304 and perpendicular to the x-axis to achieve pitch 302.

The soft robot may comprise top and bottom robot platforms further comprising flexible nitinol stents. Soft robot may further comprise compliant joints. As an example, the steerable fiber 308 may comprise various controllable materials such as electro-controllable materials, steerable catheters, or other mechanisms.

FIG. 4 illustrates the replacement of two rigid platforms 102, 110 of the 3USR robot with meshes 402, 410. These meshes may already be used in catheters to aid in forming balloon stents during surgery to treat cardiovascular conditions. Therefore, they may also be called stents, even though their function may be different. The top rigid platform 102 may be replaced with a top mesh/stent 402. The bottom rigid platform 110 may be replaced with a bottom mesh/stent 410. Similarly, the universal joints 108 may be replaced with steerable fibers 408 or with pairs of steerable fibers. The resulting device may be folded into a delivery sheath 404 or catheter.

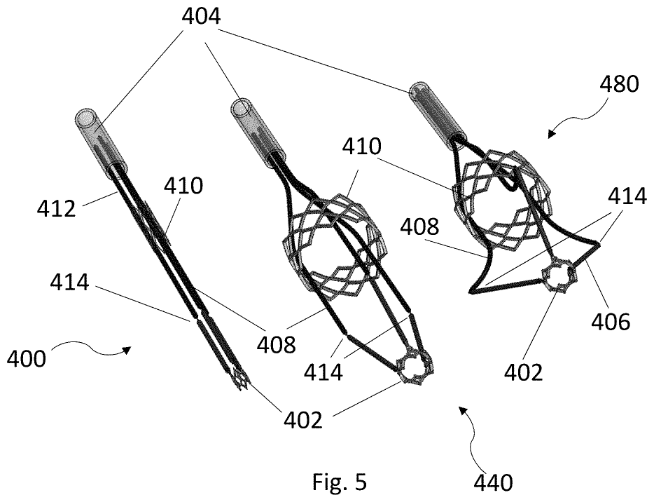

The robot structure may be soft and compliant and configured to fit inside, e.g., a catheter. FIG. 5 shows the device in several stages (e.g., folded mode (inside the catheter tube for delivery purposes), unfolded mode (inside the heart to be fully functional), and actuated mode) of the soft robot. In the folded mode 400, the device may be folded to fit into a delivery sheath 404 which may be small enough to fit into a human heart through a vein or artery. It resides inside a delivery sheath or catheter 404 for delivery into a human heart, for example. In the unfolded mode 440, the catheter has delivered the device into a heart. Steerable fibers or catheters 412 deploy to push the device out of the delivery sheath. The device then expands the upper or distal stent 402 and the lower or proximal stent 410. In this example, the proximal stent 410 expands to a greater diameter than the distal stent 402, just as in the rigid robot, the bottom platform 110 may be larger than the upper platform 102. In principal, other configurations could be used, such as the proximal stent expanding to a smaller diameter than the distal stent. In the actuated mode 480, the operator has moved or re-positioned the distal stent 402 as a platform for performing surgery. This control occurs through the use of the flexible link 408, also called a steerable catheter, and with help from a compliant link 406. The compliant link may comprise nitinol or other materials which may return to an original state after use. In addition, the device may employ soft joints 414 to connect various elements especially between a flexible link 408 and a compliant link 406.

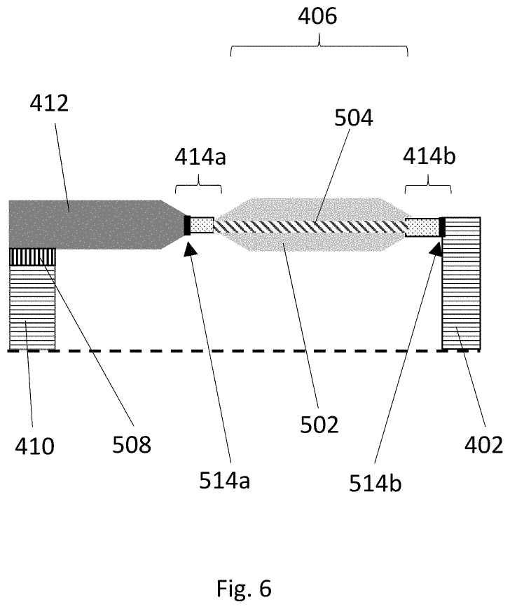

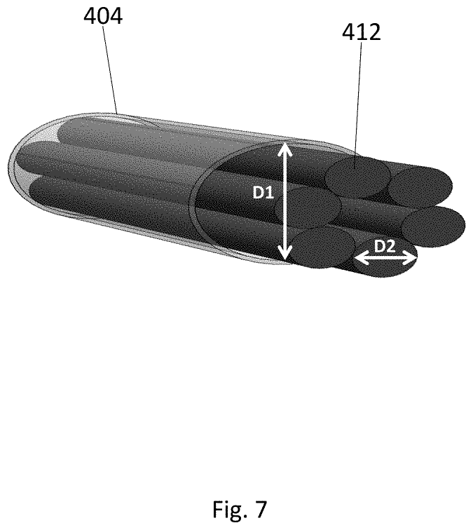

FIG. 6 and FIG. 7 further depict the device. FIG. 6 depicts a cross-section of the device. A steerable fiber or catheter 412 may be attached to the proximal stent 410 through bonding 508, such as thermoplastic bonding or adhesive bonding. The proximal soft joint 414a connects the steerable fiber/catheter 412 with a compliant link 406. The compliant link 406 may comprise a nitinol tube 502 and nylon thread 504 in the nitinol tube. The distal end of the compliant link may connect with the distal stent 402 through a second soft joint 414b. The lower soft joint 414a may be connected through a knot or thermoplastic bonding 514a to the steerable catheter. Likewise, the distal soft joint 414b may be connected to the distal stent 402 through a knot or thermoplastic bonding 514b. FIG. 7 shows a delivery sheath 404 containing steerable fibers/catheters 412. For clarity, only a single steerable catheter 412 is indicated, while the example of 6 steerable catheters are illustrated. FIG. 7 also shows the delivery sheath 404 having an internal diameter D1 and a steerable catheter 412 having an outer diameter D2. For use within a human heart, the internal diameter D1 may be approximately 10 mm and the outer diameter D2 may be approximately 2 mm.

Appropriate materials may be utilized to miniaturize the soft robot to fit within a catheter for use during human heart surgery. Two nitinol networks may function as top and bottom platforms. These nitinol networks may look and act similar to mesh stents used in other operations. Three steerable active links (e.g. size 6 French catheters) may be employed for one version. The robot components may be attached together through a thermoplastic bonding mechanism or application of UV curable plastic bonding medical device adhesive, though other methods may be also envisaged. Noting that the microcatheters may be fabricated with a typical size of 6 French (2 mm outer diameter) or smaller. The full transcatheter robot therefore may comprise several such microcatheters for links in addition to other components. Such a soft robot may be manufactured to be less than 10 mm diameter in the folded mode (see FIG. 7).

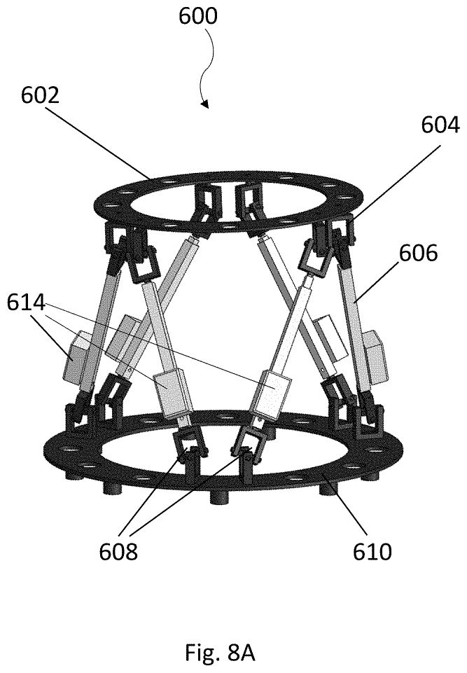

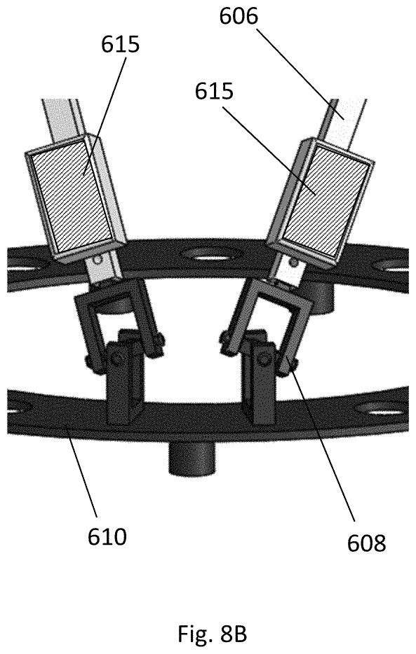

FIGS. 8A-8B show a Stewart mechanism 600. A Stewart mechanism may be known for its use in flight simulators. A Stewart mechanism 600 may have 6 degrees of freedom and can move the upper platform 602 in both the x, y, and z directions and the three angular orientations, roll, pitch, and yaw. A Stewart mechanism 600 comprises a rigid upper platform 602, six joints linking the rigid upper platform with six legs 606, and six linear actuators 614, one attached to each leg 606. In this example, the legs may telescope into themselves, allowing the linear actuators 614 to extend or retract each leg 606 independently of the others. In an alternative configuration a Stewart mechanism may act as a controller for the soft robot, much as a joystick. In such a configuration, the linear actuators 614 would be swapped out with linear potentiometers 615 to measure the displacement of each leg (see FIG. 8B). Each leg 606 may be connected to the bottom rigid platform 610 through a universal joint 608 which can rotate around two axes. FIG. 8B shows additional details of the universal joints 608 of a Stewart mechanism, especially the two axes of rotation available to each leg 606 where it may be joined to the bottom platform 610. FIG. 8B also shows that the linear actuators 614 may be replaced by linear potentiometers 615 to measure displacement of the legs. In concept, a Stewart mechanism may be similar to the 3USR robot and may be analogous to the flexible soft transcatheter robot.

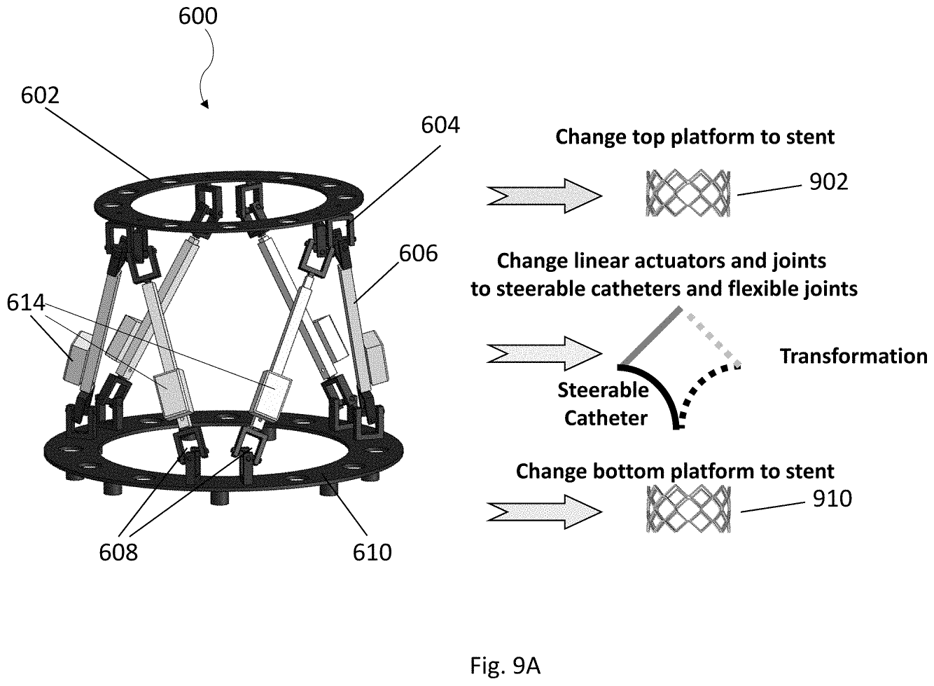

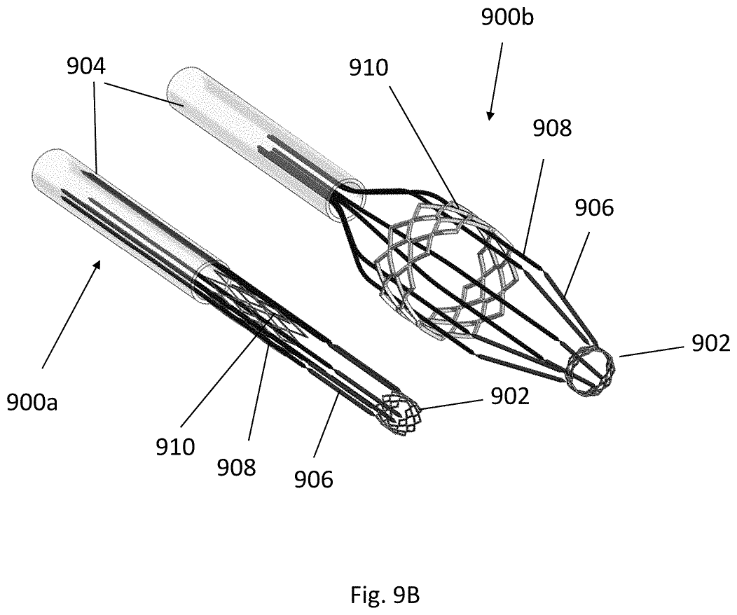

FIGS. 9A-9B show an example conversion of the Stewart mechanism 600 into a flexible soft transcatheter robot in accordance with the present disclosure. The flexible soft transcatheter robot is shown in folded 900a (e.g., inside the catheter) and unfolded mode 900b (e.g., inside the heart), and may comprise a catheter tube 904 for delivery and one or more compliant links 906 (e.g., nitinol). Although the rigid platforms 602, 610 are show being replaced by stents 902, 910 in the transcatheter conversion, other soft robots may be generated without replacing the platforms 602, 610. For example, the platforms 602, 610 may remain rigid, but the linear actuators and joints may be converted to steerable mechanisms (e.g., steerable catheter 908) and flexible links or joints.

The overview of design process, and the components of the user interface system are illustrated in FIGS. 8A-8B. A Stewart mechanism 600 may be utilized as a joystick, since it has 6 DOF, and may act as a master system for the soft robot. The kinematics of the Stewart mechanism may be used to obtain its size based on the required workspace. The structure of the mechanism may be 3D printed and assembled. Linear potentiometers 615 may be mounted on each leg 606 of the Stewart mechanism 600 to sense its displacement. Accurate measurements of these displacements may translate the surgeon's hand motion to its equivalent motion in the sensing legs that must be mapped to the required bending motions in the active links of the soft robot. After fabrication, the soft robot may be calibrated and nonlinearly mapped based on the bending profile of the soft robot's legs. This calibration data may be used as a desired input motion of the soft robot. The position of the robot may be sensed by an EM tracker attached to the robot end-effector which may be mapped to the equivalent bending deformation of the robot legs through the kinematics model and feedback to the controller.

The performance of the joystick may be validated. The joystick may be moved and rotated in all 6 DOF and the soft robot must perform accordingly. This may be verified both optically, and through the collected data from EM tracker. A Matlab program may be utilized to generate the desired 3D trajectories within the robot workspace. The program may be configured to read and plot the sensory data of the robot end-effector position online. Next, the operator may use the joystick to move the robot and follow the desired trajectory by looking at a computer monitor. The efficiency of this process in terms of tracking error and training time for using the joystick may be compared with other existing steerable catheter systems.

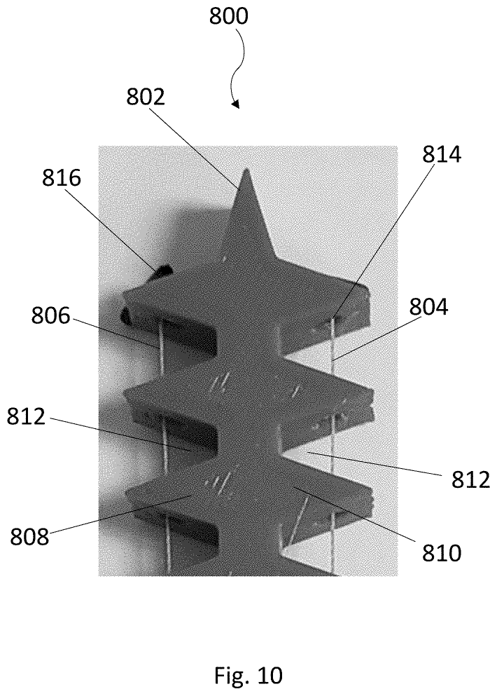

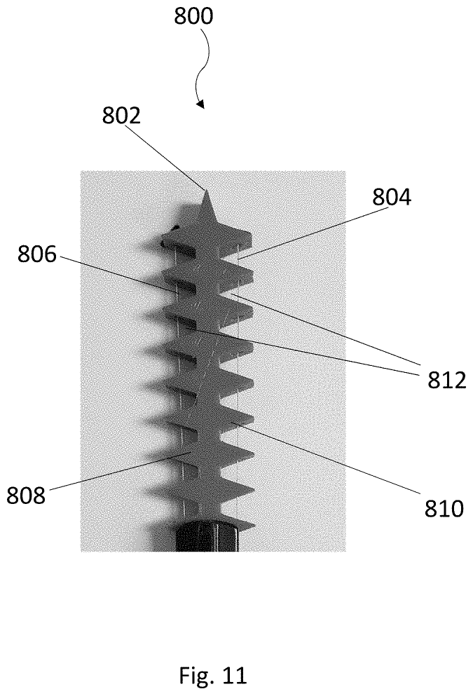

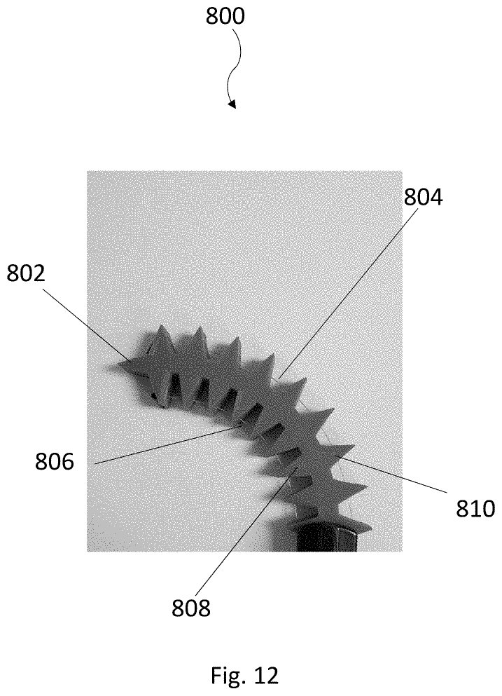

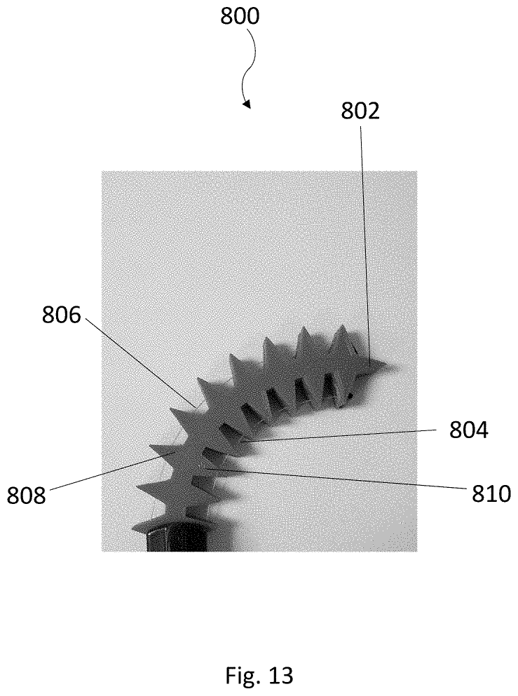

FIGS. 10-13 show examples of a soft joint 800 according to the present disclosure. The tip 802 of a soft joint is shown along with left side ridges 808 and right side ridges 810. These ridges 808, 810 leave spaces 812 for flexing on both sides. In each ridge 808, 810 may be a hole 814 through which may be threaded a cable or fiber 804, 806. The soft joint 800 has a left side cable 806 and a right side cable 804. For this proof of concept model, the left side cable 806 terminates at the top-most ridge with a nut or block 816 to prevent it from sliding out. A similar block exists on the right side (not shown in the FIGS). Alternatively, the left cable 806 and the right cable 804 may be joined or otherwise affixed atop this soft joint 800. FIG. 11 shows the full soft joint 800 when no tension is applied to the cables 806, 804 or when the tension may be roughly equal between the cables. FIG. 12 shows the soft joint 800 when more tension is applied to the left cable 806 than to the right cable 804. The left side ridges 808 may be drawn closer to each other into the spaces 812. The right side ridges 810 become further separated. FIG. 13 shows the same effect but with tension applied to the right cable 804 rather than to the left cable 806. Such a joint 800 may be fabricated by various means and operates as a tendon joint.

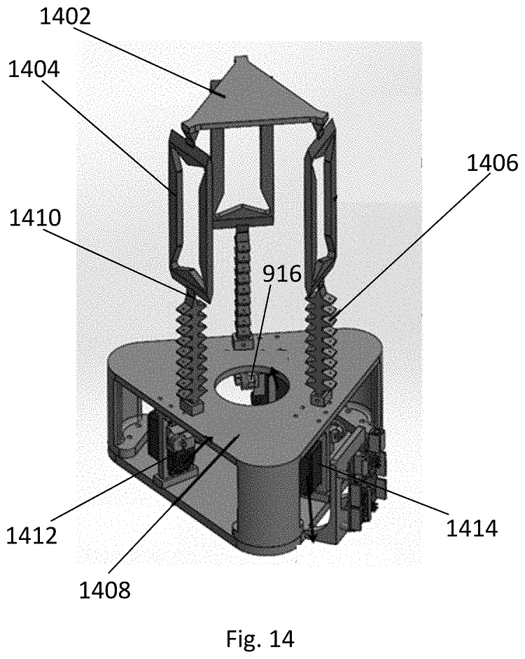

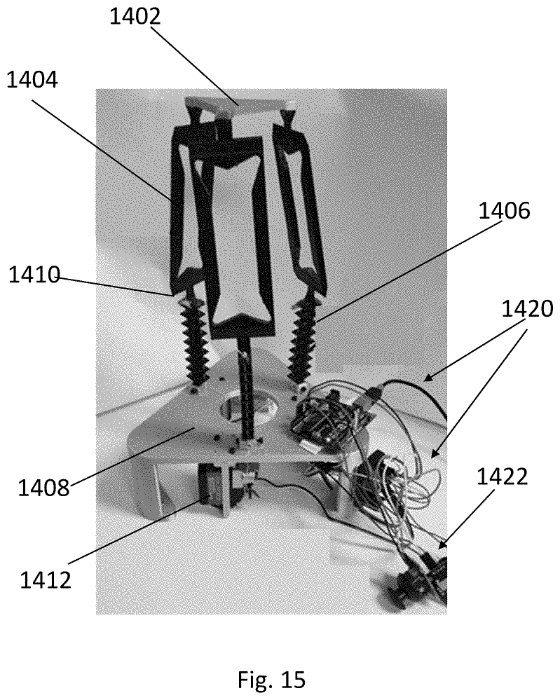

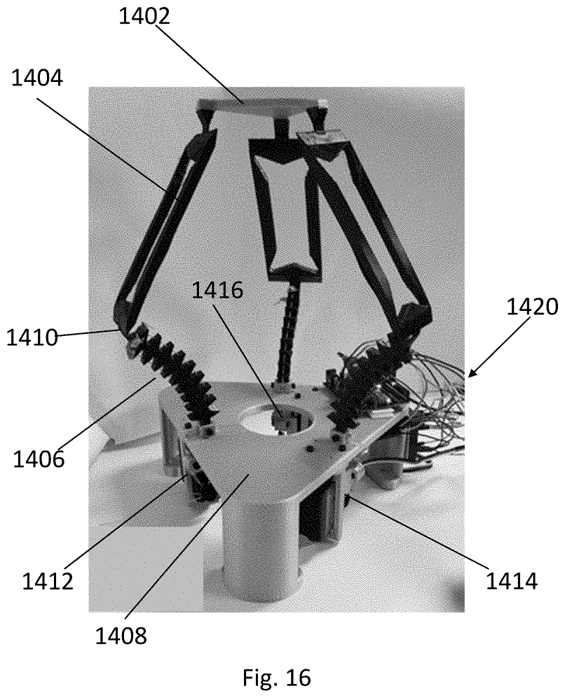

FIGS. 14-16 show a robot with soft joints and 6 DOF. This example may otherwise be similar to a Stewart mechanism or the 3USR robot, but fabricated with soft joints. FIG. 14 illustrates a CAD drawing of such a robot. The upper platform 1402 may be connected to the lower platform 1408 by soft joints 1406. Three motors 1412, 1414, and 1416 may rotate to apply tension to the fibers which control the soft joints 1406. A flexure hinge 1410 may connect the soft joint 1406 with the concentrated compliant four bar 1404 upon which the upper platform 1402 rests. FIG. 15 shows the proof-of-concept robot with the soft joints 1406. In addition to the elements from FIG. 14, control electronics 1420 and at least one joystick 1422 for controlling the top platform may be employed. FIG. 16 shows the same device with soft joints flexed to demonstrate translational control of the upper platform 1402. With each of the soft joints 1406 contracted outwards, the upper platform 1402 may be lowered.

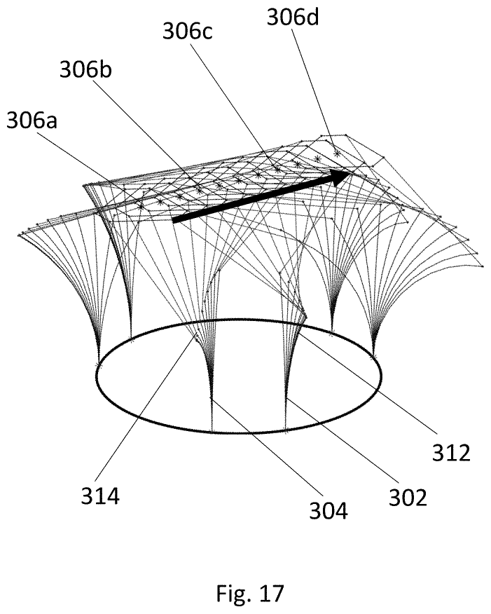

If the motor strings are actuated in the same direction, then the top plate may be displaced by up to 13 cm for the demonstration unit. The strings may be pulled as a function of time to obtain all possible configurations of the soft robot as illustrated in the simulations described below and in FIGS. 17-19.

The soft bottom links may be actuated by three servo motors as the CAD model and initial prototype show in FIGS. 15-16. Each pair of soft and concentrated compliant four-bar links may be designed monolithically and 3D printed as a single piece in thermoplastic polyurethane (TPU). The soft links may be supported by the base which houses the three actuators. The actuators may be controlled by three joysticks 922 (only a single joystick 922 is visible in FIG. 15) and based on the input from the user, the soft bottom links bend or flex depending on which way each tendon joint is actuated. If all three links are provided the same input, the top plate may move up or down while keeping it parallel to the support base as demonstrated in the expanded form of the robot in FIG. 16.

The design and fabrication of the soft robot provides 6 DOF motion. In some embodiments, there may be four sections of the soft link arms. The bottom of the arm connected to the base may be a compliant flexure with a height of 90.5 mm and a thickness of 6 mm. (FIG. 14 shows the CAD figure for this simulation.) There may be nine protruding triangular ridges on both sides of this section where the flexure will bend inwards and outwards. (i.e., as shown in FIGS. 10-13) There may be a small opening down the face of all ridges where a cable passes through to deform the soft links. Two cables may be connected to the top ridge to bend the soft links in two directions through the servo motors. This allows both inward and outward movements depending on which cable is pulled down, with the corresponding cable being released from tension. The soft arm may include soft and compliant links which have a height of 120 mm and a width of 1.5 mm possessing flexure hinges at the edges. The compliant flexures allow the links to bend and revolve depending on the actuation.

For simplicity, the protruding triangular regions are omitted in the Simscape model. The superiority of the Simscape modeling of soft robots may be that any data can be exported if the corresponding sensors are utilized in the model. Rigid Transform blocks may be used to position the placements of the cable while a brick solid block may be connected to the top of the general flexible block that will be used to tie the ends of the cable. This brick solid block has the same distance across the small holes in the arm's first region ridges and to connect the cable to the brick solid ends, frames may be introduced in the settings of the brick solid block. A belt-cable end block may be used to attach the cable to this frame. As a first test, the top cable on both sides of the brick solid may be pulled down. To model the pulley mechanism, the belt-cable end may be connected to a pulley block located at the bottom of the general flexible block. The bottom pulley may be located at a distance by using a rigid transform block. The rigid transform block may be connected to a revolute joint to rotate the pulley along with a cylindrical solid that must be used for the pulley wheel. The revolute joint and the cylindrical solid may also be connected to a belt-cable spool which represents the pulley.

The soft robot may comprise three flexible links 3D printed using thermoplastic polyurethane (TPU) which may comprise an active soft link and a compliant four-bar linkage connected with flexure as depicted in FIGS. 15-16. The fin-like soft link with the strings may allow the link to deform in either direction once the string is actuated as shown in FIGS. 10-13. If the string running on both sides of the soft link is pulled/actuated thereby releasing the right string, the link deformed to the left. Likewise, once the right string is actuated and the left string is released, the soft link may deform to the right as expected. The fins on the soft link may prevent the pulling of the soft link more than a certain degree. The soft link may comprise a plurality (e.g., 8) fins with a length of 110 mm and an overall width of 26 mm.

The compliant four-bar link may enable the tip of the soft link-compliant four-bar arm to move side to side without bending the soft link. The soft link and the compliant link may be connected through a compliant flexure with 10 mm thickness to create relative motion between each.

The soft robot may comprise 3 flexible links, three servo motors with strings, a top platform, a bottom platform that houses the servo motors, and a microcontroller (e.g., Arduino), as shown in FIGS. 14-16. The soft links may be 3D-printed using TPU and the top and bottom platforms housing the servos may be 3D-printed out of polylactic acid (PLA). The three soft links may be attached on the bottom platform by 120° apart from each other as well as 60.3 mm apart from the center of the bottom platform. Also, the soft links may be connected to the top platform which may be kept parallel to the bottom platform in its initial configuration. Each of the flexible links had embedded strings that may be actuated by a pulley wheel connected to a servo motor. The servo motors may be connected to an Arduino and can be controlled independently via the joysticks or computer.

If all the links are actuated in the same direction, then the top plate follows a straight up and down motion while the top plate remains parallel to the ground. Even though the top platform may be lowered due to the outward deflection of the soft links, the displacement may not be significant since the string got loose after a couple of cycles due to the connections at the top end of the fin-like structure and the lack of tension. In addition, the compliant four-bar linkage is too stiff and prevented the required side to side motion. Consequently, the soft links bent side-to-side as opposed to bending along the direction in which the strings are actuated.

To address these issues, an initial proof-of-concept design was modified. Taking into consideration the lack of tension in the strings as well as the flexibility and stiffness, the compliant four-bar linkage may be revised from having sharp corners to curve corners with a smaller thickness. The soft link thicknesses may be increased from 10 mm to 14 mm to prevent excessive bending. Also, in order to resolve the lack of tension in the strings, a pulley wheel may be designed to tighten the string to the servo motor as well as incorporating a spring attached to both strings. This allows the servo to pull the string more effectively while adding more tension. In addition, the connection of the strings at the top ends of the soft robot may be changed from a permanent connection to an adjustable connection via a bolt and a nut. The bolt and the nut may be wrapped by the string added more tension to the string while allowing it to be adjusted if it became loose under actuation. After making the necessary adjustment to the soft delta robot, the robot was reassembled with the modified soft links and the pulley wheels. Additionally, the mechanism may be controlled by programming via the computer instead of the joysticks.

The soft delta robot experiment setup may include of the soft delta robot, power supply, a laptop with MATLAB and Arduino IDE, and an ultrasonic sensor with an Arduino. The soft delta robot may be tested for up and down and side to side motion to ensure that the top plate remains parallel to the bottom platform as well as testing the performance and behavior of the soft link. An ultrasonic sensor may be used to measure the distance at which the top platform moves down. As the soft link moves outward, the top platform may be displaced 12 cm in the z direction.

Although soft robots provide more advantages compared to rigid mechanisms, their modeling is much more complex since soft link deformations yield nonlinear equations. Several approaches have been adopted in the literature. While the Cosserat rod theory may be used for the modeling of tendon-driven soft robots, modeling through finite element analysis may be the most common method among many since it provides an accurate solution considering the material nonlinearity.

Each kinematic chain connecting the top plate to the support base may be designed as a soft link connected to a compliant four-bar linkage through soft joints. The deformation of the links may be obtained from the kinematic model. Dynamical modeling through the Simscape model may be the very first soft robot modeling.

The initial design may include three main parts: a base (bottom or fixed platform), soft links, and top plate (movable platform). Once a new Simscape model is created using the command smnew, the blank model may open a default window with world frame, solver, and mechanism configuration blocks. The gravitational force can be modified using the mechanism configuration block depending on the configuration of the design. A general flexible block may be selected from the Simulink library to connect these three blocks.

The soft robot may also comprise two main body parts which may be connected through soft links and compliant joints. Any mechanism that has the tendency to bend is considered a compliant mechanism; in other words, the input force, torque, or displacement is transferred from one point to another point on the mechanism through the deformation of its flexible members rather than the revolute motion between the rigid links. Fully compliant mechanisms do not involve any joints and may be designed as a single piece. These types of mechanisms are generally limited to small ranges of displacement. Semi-compliant mechanisms include both compliant parts and traditional rigid links and joints. Compliant mechanisms can accomplish motions that may be difficult and expensive to achieve by rigid-body mechanisms by exploiting the flexibility of their components. The advantages of compliant mechanisms over traditionally designed rigid mechanisms include simplified design and improved performance. Simplified design implies a reduced number of links, lowers cost, and simplifies manufacturing processes, whereas improved performance can be achieved by lowered friction, larger deformations, reduced wear, and reduced lubrication requirements (or none at all). Compliant mechanisms have been receiving increasing attention due to their inherent properties and monolithic manufacturability in micro and nanoscale. Additionally, light-weight systems are desired in aerospace applications, and so distributed or lumped compliant designs serve as good candidates exhibiting high performance.

Lamina emergent mechanisms may be composed of compliant mechanisms that bend out of their plane when subjected to loading and can be designed with integrated actuators which have many applications in the aerospace and medical robotics field. Compliant mechanisms can be manufactured by injection molding; complex and high-quality creative designs can be created using single materials and multi-material additive manufacturing. Concentrated and distributed compliant mechanisms have been widely designed for accurate positioning, adjustment, and manipulation. For monolithically designed systems, links may be connected to each other in a materially coherent way. To create relative motion for monolithic compliant mechanisms; revolute (hinge), prismatic, or screw pair joint types may be utilized. The flexure hinges may be considered as the revolute joints in a rigid body mechanism. Flexure hinges may be designed as, for example, a leaf, cross-leafed, prismatic crossed, notch, or butterfly hinge. Typical geometries for notch type flexure hinges may be circular, corner-filleted, elliptical, and variable power function-based contour.

In an initial design phase, a 3D printed prototype of the soft robot may be designed and manufactured. Further optimizations and design validations may be also carried out. The final product may be fabricated using nitinol stents and steerable catheters. A first step in design of the 3D printed prototype may be to derive the kinematics equations, next these equations may be used to optimize the size of robot components so that it satisfies the design objectives in terms of workspace and required DOF. A first proof-of-concept unit may be fabricated using 3D printed parts, but may be too large to fit within a catheter with an inner diameter of 10 mm. Next, the dynamics of the robot may be derived and simulated using MATLAB. The dynamics equations of the robot may be used to design a controller using Quantitative Feedback Theory (QFT) for robust position control of the robot. QFT may be one of the most powerful robust control techniques which can take into account both parameter and structure uncertainties. Horowitz initially introduced QFT as a robust feedback control design technique. This technique has been further developed by Horowitz and others. In most frequency domain robust control methods such as H_{∞} (H-infinity) the controller design procedure may only be based on the magnitude of the transfer function. However, QFT not only takes into account the magnitude information but also considers the phase information in the design process. The main advantage of the QFT method may be that it can allow direct controller design based on robust performance bounds.

The effect of parameters such as friction and external disturbances due to the pulsatile blood flow make a dynamics model of the catheter highly nonlinear and uncertain. Thus, it may be very difficult to obtain robust stability and tracking performance for a soft robot system. To solve this problem, the nonlinear uncertain dynamics of the soft robot may be replaced with a family of linear time-invariant (LTI) models with parameter uncertainty. Next, a robust controller based on QFT may be designed for robust position control of the robot. The QFT controller control design procedure can be summarized as follows: Firstly, the variation of the system parameters at fixed frequencies will be obtained (plant template). Secondly, the robust performance bounds will be calculated based on the design requirement and the templates.

The designed soft robot may comprise a top platform, a bottom platform, at least one compliant link (or a set of compliant links), an actuation module and at least one position sensor (and preferably a group of position sensors). The actuation module comprises DC electromotors and tendon driven actuators which resemble steerable catheters. Without loss of generality a soft parallel robot with 3 DOF which resembles the Delta robot may be designed and fabricated. In the second phase of design, once the size of the bottom platform, top platform, active links, and passive links of the soft robot may be identified through kinematics analysis, the performance of the robot may be validated through numerical simulations and 3D printed models.

As mentioned, the soft parallel robot may have 3 DOF. The Delta robot has a similar structure to the 3USR robot but has only 3 DOF (translational motions in x, y, and z axis).

The degrees of freedom of the robot can be calculated based on the following equation:

DOF=6(N−1)−NR(5)−NU(4)−NS(3) (1)

where N is the total number of links, NR is the number of revolute joints, NU is the number of universal joints, and NS is the number of spherical joints. The proposed soft robot may comprise 8 links, three revolute joints, three universal joints, and three spherical joints. Thus, the soft robot has six DOF as follows:

DOF=6(8−1)−3(5)−3(4)−3(3)=6 (2)

Therefore, this design provides a high level of maneuverability for 3D transcatheter interventions.

In order to design the structure of the robot and analyze its workspace, a kinematic model of the robot may be developed. A constant curvature for the motion of the 3D printed tendon-driven actuators may be assumed. Next, proper frames may be assigned to the soft robot platforms. Based, on a kinematics model of the soft robot can be simulated using such equations as follow:

{Bᵢ^{B}}+{Lᵢ^{B}}+{lᵢ^{B}}={P_{P}^{B}}+[R_{P}^{B}]{Pᵢ^{P}}i=1,2,3. (3)

where {Bᵢ^{B}} is the position of the vertex of robot fixed platform, {Lᵢ^{B}} is the position of the soft actuators, {lᵢ^{B}} is the position of the passive links, {P_{P}^{B}} is the position of the end-effector, [R_{P}^{B}] is the rotation matrix, and {Pᵢ^{P}} is the position of the vertex of moving platform. The eq. 3 states that the length of the passive links must be constant and equal to 1.

lᵢ=∥lᵢ^{B}∥=∥{P_{P}^{B}}+[R_{P}^{B}]{Pᵢ^{P}}−{Bᵢ^{B}}−{Lᵢ^{B}}∥i=1,2,3. (4)

To avoid the square-root in the norms of eq. 4 square it to get:

lᵢ²=∥lᵢ^{B}∥²=lᵢₓ²+l_{iy}²+l_{iz}²i=1,2,3. (5)

Equation 5 can be solved numerically to obtain the value of required bending angle in the active links for a given position of the robot end-effector.

The basic idea in modeling the active soft links is to consider them as a discretized system which includes rigid finite elements (RFEs) connected by spring—damping elements (SDEs). Once the simulation results are confirmed with, e.g., an experimental setup, the soft links may be integrated into the rest of the mechanism. Four revolute joints may be utilized to imitate the compliant flexures on each passive links. To this end, each of the active soft links may be modeled using four segments connected through serial revolute joints and connected to the pulley. The passive soft links may also be joined to the passive compliant links thereby increasing the simulation time. To address this problem, a general flexible beam with TPU properties may be replaced with a segmented link connected via revolute joints with the spring constant K having the same load-deflection behavior as the general flexible block. This method may enable a faster simulation response and also increase the accuracy of the dynamic solution.

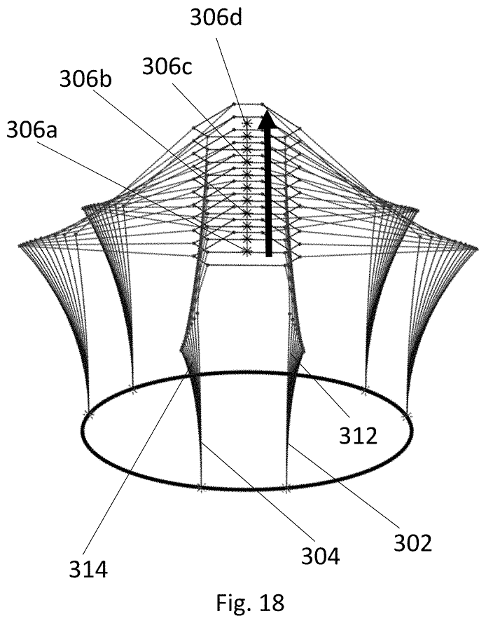

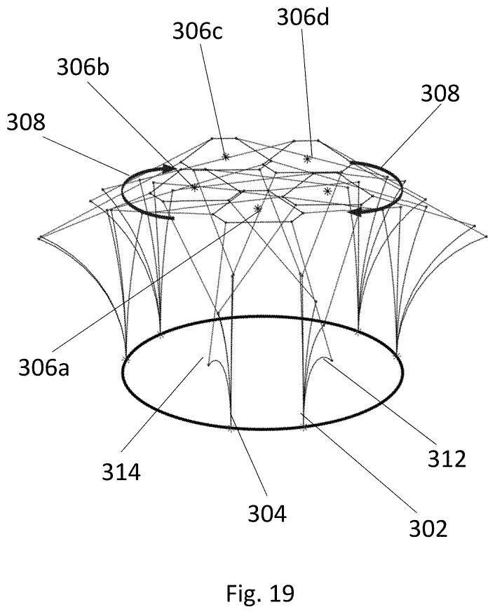

FIGS. 17-19 show the results of computer simulations of an exemplary device in actuated mode 480. The computer simulations demonstrate that horizontal motion (FIG. 17), vertical motion (FIG. 18), and circular motion (FIG. 19) may be achieved. Each of these figures illustrates 6 legs, but only two of the legs 304, 302 are identified for clarity. The legs 302, 304 correspond with the steerable fibers 408. The asterisk represents the center of the upper plate or distal stent. By varying the bending angles 312, 314 of the legs, the center of the upper plate may be propelled forward in a horizontal direction indicated by the black arrow and by the location at different times 306a, 306b, 306c, and 306d.

FIGS. 17-19 show the simulation results of the kinematics model. FIG. 17 shows for a horizontal trajectory of an object in the center of the movable (top) platform plus the required bending angles of the 3D printed soft actuators. FIGS. 18 and 19 similarly show the simulation results a horizontal trajectory and a circular trajectory, respectively.

Although the simulation time would significantly be affected, as an alternative to the creation of the design using the Simulink library blocks, the CAD model of the mechanism can be imported into Simscape and the soft links and flexures can be replaced with the flexible beams and torsional springs. The compliant four-bar linkage can also be modeled using pseudo rigid body modeling by replacing each flexure with their equivalent torsional stiffnesses having the same load-deflection behavior.

This macro scale soft robot may be fabricated to better understand the behavior of the soft links under wire actuation and serve as a proof of concept for the smaller version intended for use in surgery.

If each link is actuated at a different angle, then the top plate can possess 3 DOF. The results on the wire actuated soft robot strongly suggest that this approach yields significant behavioral improvement on the accurate positioning and manipulation of an existing robot utilized for cardiac inventions.

To experimentally validate that the robot has 6 DOF and may be capable of moving in arbitrary points within its workspace, the position and orientation of the robot will be sensed by an electromagnetic (EM) tracker attached to the robot end-effector. EM trackers have been successfully used in several studies to measure the motion of the catheter tip. The system may include two main components: sensors and an EM transmitter. The sensors may be arrays of small coils which may be placed at the catheter tip and the transmitter generates a small EM filed to track the sensors. The system may be capable of measuring 6 DOF motion (x, y, z, roll, pitch, yaw). Thus, using an EM tracker may confirm that a robot may move in x, y, and z directions and rotated in roll, pitch and yaw and thus has 6 DOF.

As has been described above, in existing steerable catheters, the roll and translational motion in the z direction may solely be due to the rotation and translation of the catheter handle. However, moving the catheter tip by rotation and translation of its handle may not be efficient due to the existence of friction and buckling. The soft robot described here solves this problem by designing so that these motions can be directly generated at the robot tip without moving its handle. Thus, the tracking error for translational motion in z direction and rotational motion about z axis (yaw rotation) may be minimized compared to other solutions.

Also, the blocking force of the robot may be measured by a sensitive force sensor and compared with a single steerable catheter. The blocking force of the robot in x, y, and z directions may be measured and compared with existing solutions for the minimum required force for cardiac interventions such as transcatheter ablation, tissue manipulation and suturing.

Ideally a joystick controller for a soft robot must have 6 DOF, be fully controlled with one hand, and be intuitive to use. Also, for convenience the workspace of the joystick must be a small volume within the range of surgeon's hand motion.

The flexible joints in the current design may be based on nylon thread that connects the steerable catheters to nitinol links and stents. Other materials may also be utilized, such as replacing the joint with a compliant spring structure made of Nitinol or other polymers. The design process may be carried out and optimized using finite element method (FEM) analysis and iterated by 3D printing.

To design a robust controller using QFT method, the desired system specifications such as tracking, and stability need to be translated into robust performance bounds that take into account the plant's uncertainty. If the robust performance bounds suggest a very high gain for the controller that results in saturation of the actuator. To solve this potential problem, the tracking bounds may be modified by adding one zero to the upper bound and one pole to the lower bound at high frequencies. Robust control effort bounds may also be introduced to limit the cost of feedback at the design frequencies and to make sure the robust controller will not wear out the actuator.

Common issues with existing catheter designs as a single steerable tube design (limited DOF, blocking force, positioning accuracy, torsional wind-up and buckling for roll and translational motions) may be solved by using a parallel robot structure. It is well known that the structure of parallel robots provides a better positional accuracy than do serial robots. (The structure of a single tube steerable catheter is analogous to a serial robot.) For parallel structures positioning errors are averaged whereas for a serial robot they may be added cumulatively. As is well known from classical mechanics the flexural stiffness of a beam (thus the amount of force it can exert) is a product of its elastic modulus and its area moment of inertia. Thus, by using a parallel structure the effective area moment of inertia of the device may be increased and as a result its blocking force may be increased compared to a single tube design. Also, the kinematics model of the robot demonstrates that it has six DOF and the rotational and translational motions may be generated without moving the base of the robot. Thus, the adverse effects of torsional wind-up and buckling will be eliminated.

Design and manufacturing of a six DOF transcatheter reconfigurable soft parallel robot inspired from the structure of the Stewart mechanism. Stewart mechanism has 6 DOF and can be used for precision surgery procedures. However, this mechanism has a rigid structure and cannot be squeezed to be fitted inside a catheter and delivered inside the heart. To solve this issue, the rigid structure of the Stewart mechanism has been transformed into a soft and reconfigurable structure that can be fitted inside a catheter using the soft robotics technology. The proposed soft robot has at least two modes or configurations: folded mode which fits inside a thin tube and unfolded mode which expands to a six DOF soft parallel robot once deployed inside the heart.

Development of an intuitive user interface for position control of the robot. The manual joint space control knobs in the existing character systems are not intuitive and require extensive training for the surgeons to understand the required knob adjustments to navigate the catheter tip. To address this issue, a master/slave system using a twin Stewart mechanism as a special joystick may be developed. This scheme may be advantageous because the robot's motion would be very intuitive, and the surgeon can control the robot end-effector just by using one hand. Thereby, the surgeon will hold the joystick in front of himself, and by simply rotating his hand to the left, the robot rotates to the left, and by moving his hand up, the robot moves up and so on. Moreover, since the robot may be reconfigurable and in its folded mode can be inserted through a small incision inside the body it can be used for a range of minimally invasive robot-assisted (MIRA) procedures such as laparoscopy, thoracoscopy, and fetoscopy.

Next, the linear actuators (electromotors) and rigid links may be replaced with active soft links (steerable catheters with one DOF) and compliant nitinol links which may be connected to stents through soft joints.

The robot must have 6 DOF, its workspace must cover a volume about the half the size of the left ventricle, it must fit inside a tube with inner diameter of 10 mm.

Linear potentiometers will be mounted on each legs of the Stewart mechanism so that it can sense its displacement. This will translate the surgeon's hand motion to its equivalent motion in the passive legs that may be mapped to bending motions in the active links of the soft robot. This will be done through calibration and nonlinear mapping based on the bending profile of the soft robots' legs. Finally, this data will be used as a desired input motion of the soft robot. The position of the robot may be sensed by EM trackers attached to the robot end-effector(s) which may be mapped to the equivalent bending deformation of the robot legs through the kinematics model and feedback to the controller.

The present disclosure comprises at least the following aspects:

Aspect 1: A device comprising: a controllable platform with multiple degrees of freedom at the distal end of a set of links; a set of flexure joints connecting the controllable platform to a second platform; a control means for moving the links so as to control the orientation and location of the controllable platform.

Aspect 2: The device of aspect 1, wherein the flexure joints are made from materials comprising thermoplastic polyurethane, or the like.

Aspect 3: The device of any one of aspects 1-2, wherein the controllable platform comprises a stent.

Aspect 4: The device of any one of aspects 1-3, wherein the links comprise nitinol.

Aspect 5: The device of any one of aspects 1-4, wherein the device is sized so as to fit into a catheter for insertion into the human body.

Aspect 6: The device of any one of aspects 1-5, wherein the catheter will fit into a human heart.

Aspect 7: The device of any one of aspects 1-6, further comprising a joystick controller for controlling the motion in 6 DOF.

Aspect 8: A device comprising: a first stent; a plurality of flexible links coupled to the first stent; a plurality of compliant links, each compliant link coupled to at least one of the flexible links; a second stent coupled to the plurality of compliant links, wherein the plurality of flexible links and the plurality of compliant links are configured to steer one or more of the first stent and the second stent in a plurality of degrees of freedom.

Aspect 9: The device of aspect 8, wherein the flexible links are made from materials comprising thermoplastic polyurethane.

Aspect 10: The device of any one of aspects 8-9, wherein the compliant links comprise nitinol.

Aspect 11: The device of any one of aspects 8-10, further comprising a catheter tube, wherein the device is configured to pass through the catheter tube in first state and to expand outside of the catheter tube in a second state.

Aspect 12: The device of any one of aspects 8-11, wherein at least a portion of the device is sized to fit into a human heart.

Aspect 13: The device of any one of aspects 8-12, further comprising a joystick controller for controlling the motion of one or more of the first stent and the second stent in six degrees of freedom.

Aspect 14: The device of any one of aspects 8-13, further comprising a controller for controlling the motion of one or more of the first stent and the second stent in six degrees of freedom, wherein the controller uses quantitative feedback theory.

Aspect 15: A device comprising: a first platform; a plurality of flexible links coupled to the first platform; a plurality of compliant links, each compliant link coupled to at least one of the flexible links; a second platform coupled to the plurality of compliant links, wherein the plurality of flexible links and the plurality of compliant links are configured to steer one or more of the first platform and the second platform in a plurality of degrees of freedom.

Aspect 16: The device of aspect 15, wherein the flexible links are made from materials comprising thermoplastic polyurethane.

Aspect 17: The device of any one of aspects 15-16, wherein the compliant links comprise nitinol.

Aspect 18: The device of any one of aspects 15-17, further comprising a controller for controlling the motion of one or more of the first platform and the second platform in six degrees of freedom, wherein the controller uses quantitative feedback theory.

Aspect 19: A method of making a device comprising: providing a first stent; providing a plurality of flexible links coupled to the first stent; providing a plurality of compliant links, each compliant link coupled to at least one of the flexible links; and providing a second stent coupled to the plurality of compliant links, wherein the plurality of flexible links and the plurality of compliant links are configured to steer one or more of the first stent and the second stent in a plurality of degrees of freedom.

Aspect 20: The method of aspect 19, wherein the flexible links are made from materials comprising thermoplastic polyurethane.

Aspect 21: The method of any one of aspects 19-20, wherein the compliant links comprise nitinol.

Aspect 22: The method of any one of aspects 19-21, further comprising: providing a catheter tube, wherein the device is configured to pass through the catheter tube in first state and to expand outside of the catheter tube in a second state.

Aspect 23: The method of any one of aspects 19-22, further comprising: providing a joystick controller for controlling the motion of one or more of the first stent and the second stent in six degrees of freedom.

Aspect 24: The method of any one of aspects 19-23, further comprising: providing a controller for controlling the motion of one or more of the first stent and the second stent in six degrees of freedom, wherein the controller uses quantitative feedback theory.

What is claimed is:

1. A device comprising:
a first stent;
a plurality of flexible links coupled to the first stent;
a plurality of compliant links, each compliant link coupled to at least one of the flexible links;
a second stent coupled to the plurality of compliant links,
wherein the plurality of flexible links and the plurality of compliant links are configured to steer one or more of the first stent and the second stent in a plurality of degrees of freedom.

2. The device of claim 1, wherein the flexible links are made from materials comprising thermoplastic polyurethane.

3. The device of claim 1, wherein the compliant links comprise nitinol.

4. The device of claim 1, further comprising a catheter tube, wherein the device is configured to pass through the catheter tube in first state and to expand outside of the catheter tube in a second state.

5. The device of claim 1, wherein at least a portion of the device is sized to fit into a human heart.

6. The device of claim 1, further comprising a joystick controller for controlling the motion of one or more of the first stent and the second stent in six degrees of freedom.

7. The device of claim 1, further comprising a controller for controlling the motion of one or more of the first stent and the second stent in six degrees of freedom, wherein the controller uses quantitative feedback theory.

8. A device comprising:
a first platform;
a plurality of flexible links coupled to the first platform;
a plurality of compliant links, each compliant link coupled to at least one of the flexible links;
a second platform coupled to the plurality of compliant links,
wherein the plurality of flexible links and the plurality of compliant links are configured to steer one or more of the first platform and the second platform in a plurality of degrees of freedom.

9. The device of claim 8, wherein the flexible links are made from materials comprising thermoplastic polyurethane.

10. The device of claim 8, wherein the compliant links comprise nitinol.

11. The device of claim 8, further comprising a controller for controlling the motion of one or more of the first platform and the second platform in six degrees of freedom, wherein the controller uses quantitative feedback theory.

12. The device of claim 8, further comprising a catheter tube, wherein the device is configured to pass through the catheter tube in first state and to expand outside of the catheter tube in a second state.

13. The device of claim 8, wherein at least a portion of the device is sized to fit into a human heart.

14. The device of claim 8, wherein each of the first platform and the second platform comprises a stent.

15. A method of making a device comprising:
providing a first stent;
providing a plurality of flexible links coupled to the first stent;
providing a plurality of compliant links, each compliant link coupled to at least one of the flexible links; and
providing a second stent coupled to the plurality of compliant links, wherein the plurality of flexible links and the plurality of compliant links are configured to steer one or more of the first stent and the second stent in a plurality of degrees of freedom.

16. The method of claim 15, wherein the flexible links are made from materials comprising thermoplastic polyurethane.

17. The method of claim 15, wherein the compliant links comprise nitinol.

18. The method of claim 15, further comprising:
providing a catheter tube, wherein the device is configured to pass through the catheter tube in first state and to expand outside of the catheter tube in a second state.

19. The method of claim 15, further comprising:
providing a joystick controller for controlling the motion of one or more of the first stent and the second stent in six degrees of freedom.

20. The method of claim 15, further comprising:
providing a controller for controlling the motion of one or more of the first stent and the second stent in six degrees of freedom, wherein the controller uses quantitative feedback theory.
